(19) United States
(12) Patent Application Publication
(10) Pub. No.: US 2013/0053579 A1
(43) Pub. Date: Feb. 28, 2013

(54) PROCESS FOR THE PREPARATION OF N-METHYL-O-ARYLOXY PROPANAMINE DERIVATIVES AND PHARMACEUTICALLY ACCEPTABLE SALT THEREOF

(71) Applicants: Nishikant Digamber Ghadge, Mumbai (IN); Bapu Atmaram Chaudhari, Mumbai (IN); Ganesh Gurpur Pai, Mumbai (IN); Arun Kanti Mandal, Mumbai (IN)

(72) Inventors: Nishikant Digamber Ghadge, Mumbai (IN); Bapu Atmaram Chaudhari, Mumbai (IN); Ganesh Gurpur Pai, Mumbai (IN); Arun Kanti Mandal, Mumbai (IN)

(73) Assignee: ARCH PHARMALABS LIMITED, Mumbai (IN)

(21) Appl. No.: 13/384,757

(22) PCT Filed: Nov. 30, 2010
(86) PCT No.: PCT/IN2010/000771

§ 371 Date: Jan. 18, 2012

(30) Foreign Application Priority Data
May 18, 2010 (IN) 1557/MUM/2010

(51) Int. Cl. C07D 333/20; C07D 409/12

(52) U.S. Cl. 549/59; 549/75

(57) ABSTRACT

A process for the preparation on N-methyl-aryloxy-propanamine derivatives of the formula I and salts thereof. The invention also relates to the preparation and use novel intermediate of the formula XII. The invention also relates to the process of further conversion of novel intermediate into N-methyl-aryloxy propanamine derivatives and salts thereof.

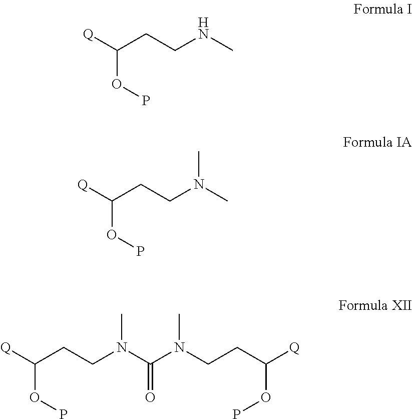

Wherein Q and P independently represents substituted or unsubstituted aryl group such as phenyl, naphthyl, pyridine, furanyl, pyranyl thienyl, and the like optionally substituted aryl by a halogen, a straight chain or branched alkyl group containing 1 to 6 carbon atoms, —O-alkyl group containing straight chain or branched C1-C6 alkyl group, an alkoxy group containing a straight chain or branched alkyl group having 1 to 6 carbon atoms, which comprises demethylation of N,N-dimethyl analogues of compound of formula IA.

### CROSS REFERENCE TO RELATED APPLICATIONS

The present application is a United States national phase application filed under 35 USC 371, which claims the benefit of priority based on Patent Cooperation Treaty (PCT) Application Serial No. PCT/IN2010/000771, filed on Nov. 30, 2010, which in turn claims the benefit of priority to India Patent Application Serial No. 1557/MUM/2010, filed on May 18, 2010, all commonly owned herewith.

### TECHNICAL FIELD OF THE INVENTION

The present invention relates to a process for the preparation on N-methyl-aryloxy-propanamine derivatives of the formula I and salts thereof. The invention also relates to the preparation and use of novel intermediate of the formula XII. The invention also relates to the process of further conversion of novel intermediate into N-methyl-aryloxy propanamine derivatives and salts thereof

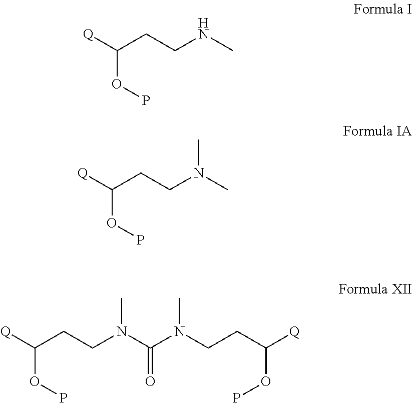

Wherein Q and P independently represents substituted or unsubstituted aryl group such as phenyl, naphthyl, pyridine, furanyl, pyranyl thienyl, and the like optionally substituted aryl by a halogen, a straight chain or branched alkyl group containing 1 to 6 carbon atoms, —O-alkyl group containing straight chain or branched C₁-C₆ alkyl group, an alkoxy group containing a straight chain or branched alkyl group having 1 to 6 carbon atoms, which comprises demethylation of N,N-dimethyl analogues of compound of formula IA. The compounds represented by formula I are widely used in medicine as antidepressants. Compound of Formula I includes racemic mixture and optical isomers thereof. The depressive or anxiety disease results from the decreased concentration of serotonine in the central nervous system, which can be compensated for either by increasing the rate of biosynthesis of serotonine on inhibiting the metabolism thereof.

### TECHNICAL BACKGROUND OF THE INVENTION

The method according to the present invention can be advantageously used for the preparation of several pharmaceutically active ingredients widely used in the medicines for the treatment of diseases or disorders of the central nervous system including the compounds selected from the series containing common N-methyl-aryloxy-propanamine structural unit such as duloxetine of formula II, fluoxetine of formula III tomoxetine of the formula IV, atomoxetine of the formula V, nisoxetine of the formula VI and the like. The compounds mentioned herein belong to the group of selective serotonine norepinephrine reuptake inhibitors (SSNRI), which decrease the rate of metabolism of serotonine by inhibiting the back flow of serotonine or norepinephrine from the receptors, thus inhibiting the rapid inactivation of the neurotransmitters.

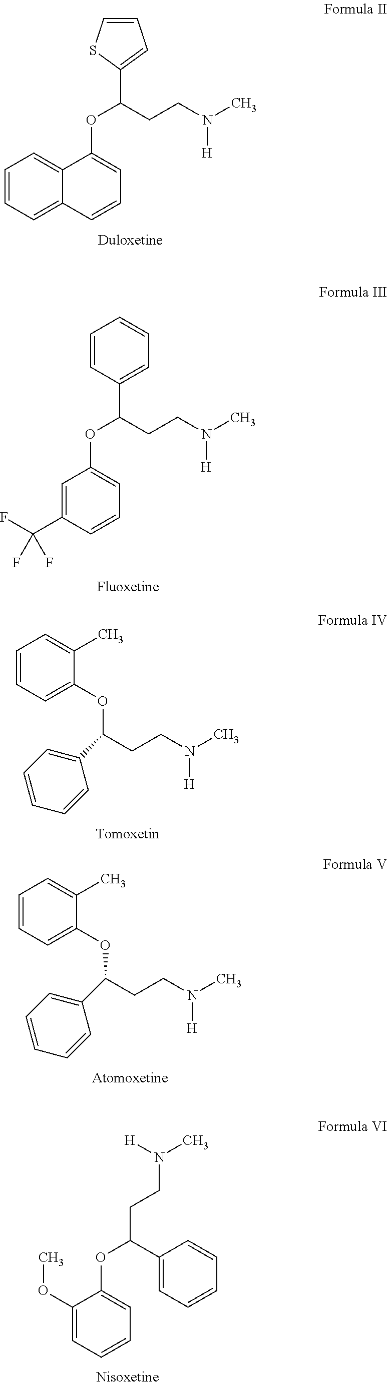 [SMILES: CNCCC(Oc1cccc2ccccc12)c1cccs1 ; CNCCC(Oc1cccc(C(F)(F)F)c1)c1ccccc1 ; CNCC[C@@H](Oc1ccccc1C)c1ccccc1 ; CNCC[C@@H](Oc1ccccc1C)c1ccccc1 ; CNCCC(Oc1ccccc1OC)c1ccccc1]

All the compounds mentioned hereinbefore possess the N-methyl propylamine structural unit which can be synthesized by several chemical methods available in the state of the art and are comprising the demethylation of the corresponding N,N-dimethyl analogues.

There are well known methods in the art for the preparation of N-methyl aryloxy propanamines. First method comprises debenzylation of corresponding N-methyl N-benzyl analogues by using catalytic hydrogenation in presence of palladium/charcoal. Second method comprises demethylation of corresponding N,N dimethyl analogues by treating them with chloroformates such as ethyl, methyl, phenyl and the like affording their carbamates as intermediates followed by their hydrolysis yielding corresponding N-methyl propylamines.

A method comprising debenzylation of corresponding N-methyl N-benzyl propanamine of formula VII with phenyl chloroformate has been disclosed in EP Patent No. 617,006 for the preparation of fluoxetine of formula III.

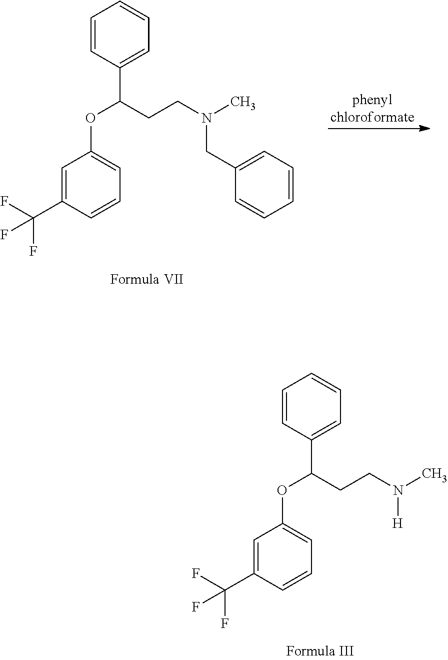 [SMILES: CN(CCC(Oc1cccc(C(F)(F)F)c1)c1ccccc1)Cc1ccccc1 ; CNCCC(Oc1cccc(C(F)(F)F)c1)c1ccccc1]

U.S. Pat. No. 5,023,269 discloses preparation of racemic duloxetine and its pharmaceutically acceptable salts comprising demethylating the corresponding N,N dimethyl propanamine derivative using phenyl chloroformate as shown herein below in scheme-1 to yield the corresponding carbamate as an intermediate. The carbamate was then hydrolysed to afford racemic duloxetine as an oil which was then isolated as oxalate salt. The process disclosed herein results in the formation of phenolic impurity which is removed by purification process to prepare pharmaceutically acceptable final product.

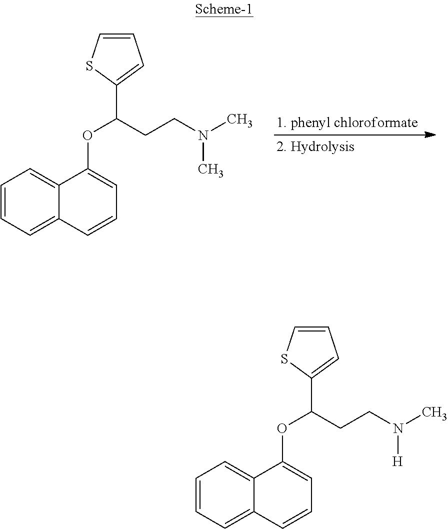 [SMILES: CN(C)CCC(Oc1cccc2ccccc12)c1cccs1 ; CNCCC(Oc1cccc2ccccc12)c1cccs1]

U.S. Pat. No. 5,362,886 hereinafter referred as '886 discloses an improved process for the preparation of (S)-duloxetine comprising reaction of (S) isomer of hydroxyl derivative of the formula VIII with 1-fluoronaphthalene in presence of sodium hydride and additional potassium salts. In '886 patent optical (S) isomer of duloxetine was prepared by demethylating the corresponding N,N dimethyl propanamine derivative using phenyl chloroformate to yield the corresponding carbamate as an intermediate which is hydrolysed to give the corresponding product as shown herein below in scheme-2.

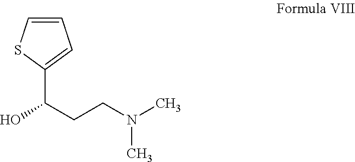

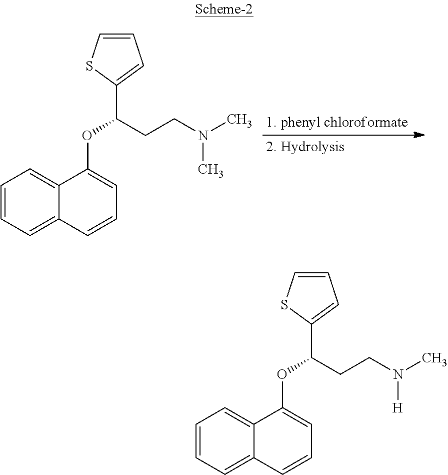 [SMILES: CN(C)CC[C@H](Oc1cccc2ccccc12)c1cccs1 ; CNCC[C@H](Oc1cccc2ccccc12)c1cccs1]

The process disclosed herein in scheme-2 results in the formation of phenolic impurity which has to be removed to obtain pharmaceutically acceptable final product.

WO2006/27798 discloses preparation of (S)-isomer of duloxetine from the corresponding N-methyl-N-benzyl propanamine analogue by catalytic hydrogenation using palladium/charcoal as shown herein below in scheme-3

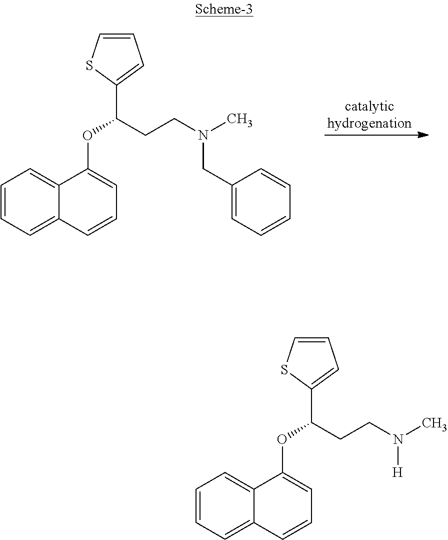 [SMILES: CN(CC[C@H](Oc1cccc2ccccc12)c1cccs1)Cc1ccccc1 ; CNCC[C@H](Oc1cccc2ccccc12)c1cccs1]

WO2007/5643(example 33 and 34) discloses the preparation of (R) isomer of deuteron tomoxetine comprising the demethylation using phenyl chloroformates as shown hereinbelow in scheme-4. The process disclosed herein results in the formation of phenolic impurity which has to be removed to obtain pharmaceutically acceptable final product.

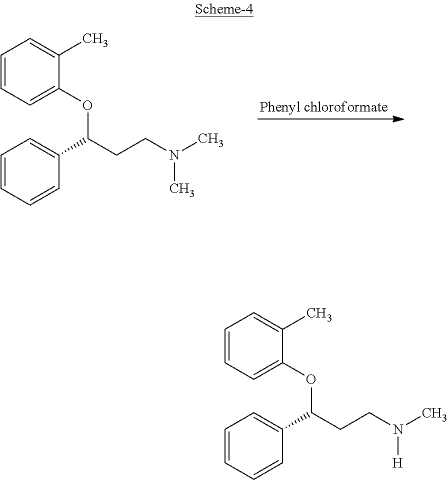

Processes disclosed therein in the prior art for the synthesis of N-methyl-propanamine derivative of the formula I wherein aryl hetero substituent is not present comprises for the preparation of said structural unit starting from N-methyl-N-benzyl propanamine analogue, which is converted into the product by removing the benzyl group by catalytic hydrogenation using palladium—charcoal catalyst.

However, in case of molecule of formula IX wherein N-methyl-propanamine is substituted by an aryl heteroatom substituent, the catalytic debenzylation of the corresponding N-methyl-N-benzyl analogue of formula X or its optical isomers may be cumbersome as the hetero atom may deactivate the palladium catalyst.

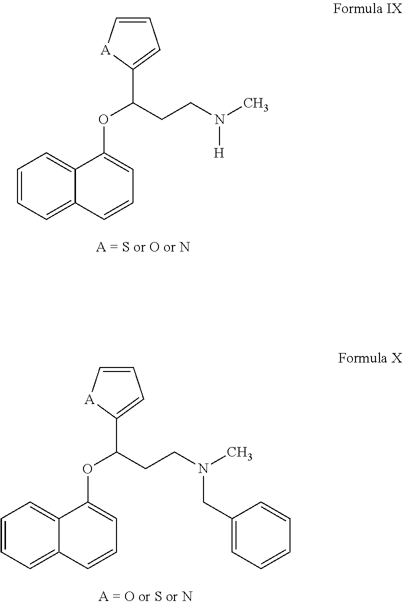

For the heteroatom containing compounds of formula IX where catalytic hydrogenation is not suitable, the N-methyl propanamine structural unit is generally synthesized from the corresponding N,N-dimethyl propanamine unit. Such starting compound containing the N,N-dimethyl-propanamine structural unit for compound of formula-IX is N,N-dimethyl-3-(1-naphthyloxy)-3-(2-aryl)propanamine of the formula XI

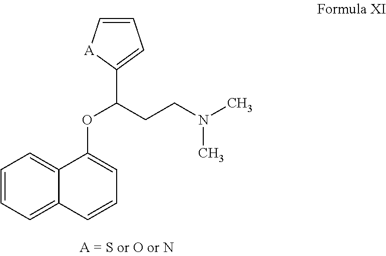

The N,N-dimethylamino group is converted into corresponding carbamates by reacting the said group with alkyl or aryl chloroformates. The said carbamates on subsequent hydrolysis in the presence of a base yield the compound of formula-IX. N,N-dimethyl amino compounds reacts readily at a temperature of 40 to 100 degree Celsius with alkyl or aryl chloroformates. Chloroformates used for the purpose disclosed therein in the prior art are methyl, ethyl(mostly used), 2,2,2-trihaloethyl, chloroformates (WO2008/004191), 1-chloroethyl chloroformate (US2009/009), phenyl chloroformate (mostly used) in presence of an acid scavenger.

It has been observed that during the conversion of carbamate obtained by reacting molecule of formula XI wherein heteroatom A is S which is used to obtain molecule of formula II (Duloxetin) with alkyl chloroformate, racemisation of the optically active duloxetine occurs at the temperature above 40° C. and it increase with rise in temperature. Therefore, use of alkyl chloroformates to convert optically active isomer of molecule of formula XI into corresponding optically active isomer of compound of formula IX is not suitable as it results into racemisation of the optical isomers thus limits their use. Moreover, hydrolysis of the carbamate intermediate of compound of formula XI to obtain a compound of formula IX results in the formation of alkyl alcohol as an impurity that is generated as a byproduct.

The drawback associated with 2,2,2-trihaloethyl chloroformates is its cost, toxicity and unstability. This brings the limitation for its use on industrial scale.

In case where phenyl chloroformate is used for the purpose of demethylation of compound of formula XI, during the hydrolysis of phenyl carbamate that is formed as an intermediate results into the formation of phenol as a byproduct having very strict environmental limit concentration for waste water and also difficult for the removal from the reaction mass. This brings a limitation over the use of phenyl chloroformate.

In view of shortcomings in the processes disclosed therein in the prior art for making compounds of formula I by demethylation of compound of formula IA there is a dire need for an improved process which does not require use of metal catalyst for hydrogenation as it can't be conveniently used for molecules of formulae X and XI and alkyl and aryl chloroformates as it results in the racemisation of optically active isomer and also produces the alcohols and phenol as impurities.

### SUMMARY OF THE INVENTION

The present invention discloses an improved process for making compounds of formula I by demethylation of compound of formula IA comprising contacting the compound of formula IA with a compound of (ClCOCl)ₙ wherein n=1 to 3 to obtain a compound of formula XII which is converted into compound of formula I as shown is scheme-5.

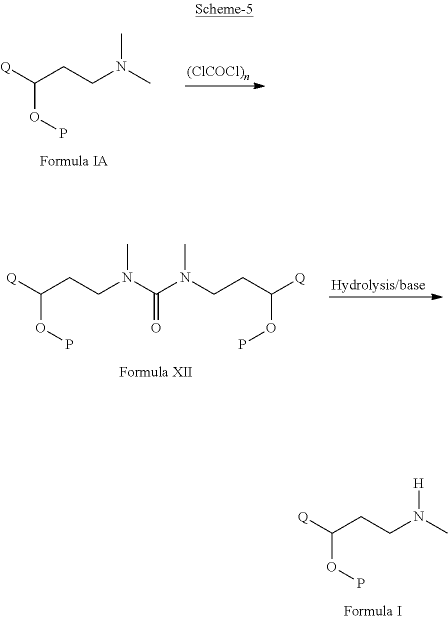

The term contacting hereinabove and hereinbelow means dissolving, slurring, stirring and the like or combination thereof.

The present invention discloses an improved process for making compounds of formula I by demethylation of compound of formula IA comprising contacting the compound of formula IA with a compd. of (ClCOCl)ₙ wherein n=1 to 3 to obtain a novel compound of formula XII which is converted into compound of formula I.

First aspect of the present invention is to provide a novel process for the preparation of N-methyl-aryloxy-propanamine derivatives of the formula I and pharmaceutically acceptable salt thereof.

Second aspect of the invention is to provide a novel intermediate of the compound of formula XII.

Third aspect of the invention is to provide a novel process comprising the demethylation of N,N-dimethyl propanamine analogue of formula IA.

Fourth aspect of the invention is to provide a method for demethylating N,N-dimethylamino-aryloxy propanamine derivatives of the formula IA comprising contacting the compound of formula IA with a phosgene molecule of formula XIII

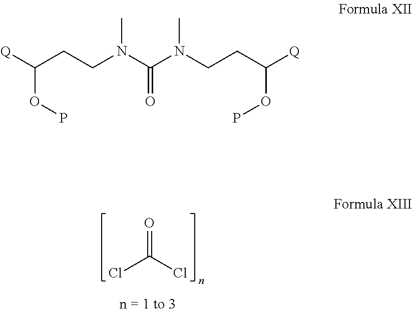

in the presence of an acid scavenger in a solvent yielding the compound which is a disubstituted urea of formula XII which on optional isolation and subsequent hydrolysis in the presence of a base gives the compound of formula I.

Fifth aspect of the present invention is to provide 1,3-dimethyl-1,3-bis(3-naphthalenyl-1-oxy)-3-(thiophenyl)propyl urea of the formula XIV as novel intermediate.

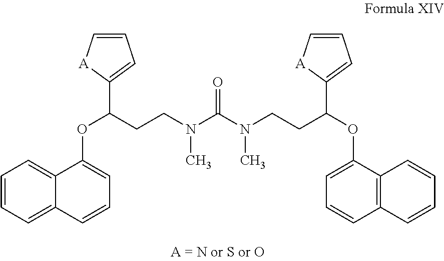

Sixth aspect of the present invention is to provide a process for the preparation of 1,3-dimethyl-1,3-bis(3-naphthalenyloxy)-3-(thiophenyl)propyl urea of the formula XIV.

Seventh aspect of the present invention is to provide 1,3-dimethyl-3-((R)-3-(1-naphthalenyloxy)-3-(thiophen-2-yl)-propyl-((S)-3-(1-naphthalenyloxy)-3-(thiophen-2-yl)-propyl)urea of formula XVI as a novel intermediate.

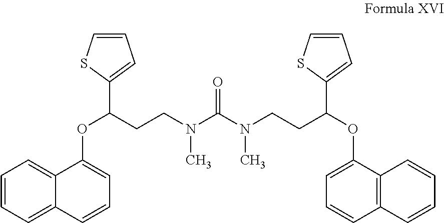 [SMILES: CN(CCC(Oc1cccc2ccccc12)c1cccs1)C(=O)N(C)CCC(Oc1cccc2ccccc12)c1cccs1]

Eighth aspect of the invention is to provide a process for the preparation of 1,3-dimethyl-3-((R)-3-(1-naphthalenyloxy)-3-(thiophen-2-yl)-propyl-((S)-3-(1-naphthalenyloxy)-3-(thiophen-2-yl)-propyl)urea of formula XIVA.

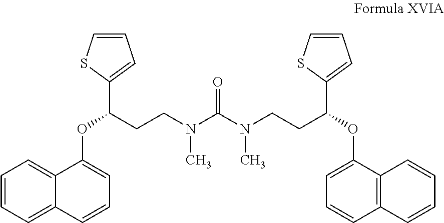 [SMILES: CN(CC[C@H](Oc1cccc2ccccc12)c1cccs1)C(=O)N(C)CC[C@@H](Oc1cccc2ccccc12)c1cccs1]

Ninth aspect of the present invention is to provide a process for the preparation of racemic duloxetine of formula II and its pharmaceutically acceptable salts comprising the making of compound of formula XVI followed by its hydrolysis using a base.

Tenth aspect of the present invention is to provide a process for the preparation of optically active duloxetine of formula IIA and its pharmaceutically acceptable salts comprising the making of compound of formula XVIA followed by its hydrolysis using a base.

Phosgene preferably triphosgene is much easier for handling on industrial scale as it is solid.

Generation of urea as a byproduct during the hydrolysis which being soluble in water can be easily removed without any extra step of purification unlike products like phenol which are difficult to remove and requires extra processing step of purification.

### DETAILED DESCRIPTION OF THE INVENTION

The present invention relates to the novel process for preparing a compd. of formula I Wherein Q and P independently represents substituted or unsubstituted aryl group such as phenyl, naphthyl, pyridine, furanyl, pyranyl thienyl and the like. Optionally substituted aryl is substituted by a halogen, a straight chain or branched alkyl group containing 1 to 6 carbon atoms, —O-alkyl group containing straight chain or branched C₁-C₆ alkyl group, by hydrolysis of a novel intermediate of formula XII which is formed by contacting compound of formula IA and compound of formula XIII. Scheme 5 given hereinbelow.

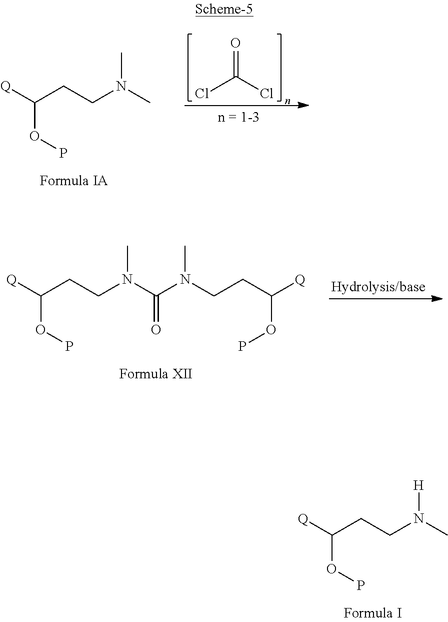

Molecule of formula XIII can be represented as:

when n=1

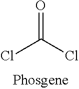

when n=2

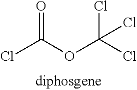

when n=3

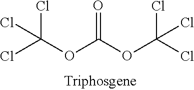 [SMILES: O=C(OC(Cl)(Cl)Cl)OC(Cl)(Cl)Cl]

In an embodiment compd of formula IA wherein Q and P independently represents substituted or unsubstituted aryl group such as phenyl, naphthyl, pyridine, furanyl, pyranyl thienyl, and the like. Optionally substituted aryl is substituted by a halogen, a straight chain or branched alkyl group containing 1 to 6 carbon atoms, —O-alkyl group containing straight chain or branched C₁-C₆ alkyl group

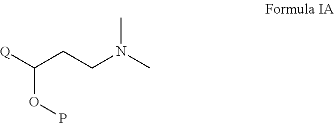

is contacted with a molecule of formula XIII

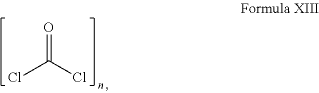

### Wherein n is 1 to 3

When n=1, the molecule of formula XIII is phosgene

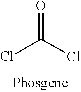

When n=2, the molecule of formula XIII is diphosgene

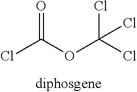

When n=3, the molecule of formula XIII is triphosgene

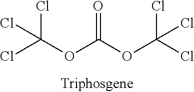 [SMILES: O=C(OC(Cl)(Cl)Cl)OC(Cl)(Cl)Cl]

in the presence of a base in a suitable organic solvent to give a compound of formula XII.

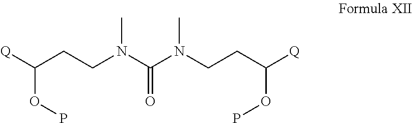

Compound of formula XIII represent phosgene, diphosgene and triphosgene. Preferably phosgene and more preferably triphosgene is used.

A solvent is selected from hydrocarbons, halogenated hydrocarbons, alcohols, ketones, esters, ethers, nitrogen atom based solvents such as triethylamine, pyridine and the likes. Preferably solvent is selected from hydrocarbons. More preferably solvent is selected from aromatic hydrocarbons. Still more preferably the solvent is toluene.

Inorganic or organic base can be used. Preferably organic base is used. More preferably ter. Amines such as trialkyl, triaryl, dialkylaryl or alkyl diaryl amines is used. Still more preferably trialkyl amine is used. Still more preferably diisopropyl ethyl amine is used.

Compd. of formula XII is contacted with a base in a suitable solvent to give a compd of formula I.

A solvent is selected from hydrocarbons, halogenated hydrocarbons, alcohols, ketones, esters, ethers, and the likes. Preferably solvent is selected from hydrocarbons. More preferably solvent is selected from aromatic hydrocarbons. Still more preferably the solvent is toluene.

Base is selected from alkali metal hydroxides, carbonates, bicarbonates, alkoxides, alkaline earth metal hydroxides and the like. Preferably the base is alkali metal hydroxides. More preferably base is potassium hydroxide.

In another embodiment compound of formula IX

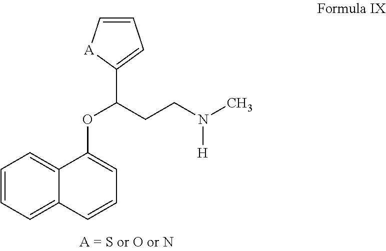

is prepared by comprising a compound of formula XI

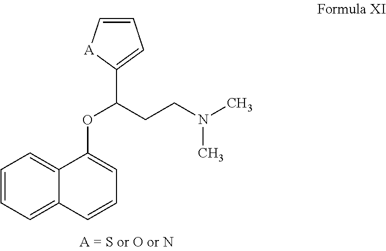

with a molecule of formula XIII Wherein n is 1 to 3

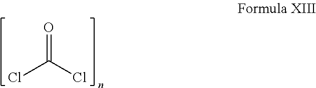

When n=1 the molecule of formula XIII is phosgene

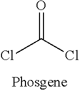

When n=2 the molecule of formula XIII is diphosgene

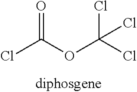

When n=3 the molecule of formula XIII is triphosgene

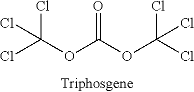 [SMILES: O=C(OC(Cl)(Cl)Cl)OC(Cl)(Cl)Cl]

in a suitable organic solvent in the presence of a base to give a compound of formula XIV

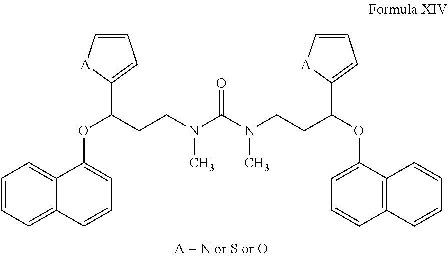

Compound of formula XIII represent phosgene, diphosgene and triphosgene. Preferably phosgene and more preferably triphosgene is used.

A solvent is selected from hydrocarbons, halogenated hydrocarbons, alcohols, ketones, esters, ethers, nitrogen atom based solvents such as triethylamine, pyridine and the likes. Preferably solvent is selected from hydrocarbons. More preferably solvent is selected from aromatic hydrocarbons. Still more preferably the solvent is toluene.

Inorganic or organic base can be used. Preferably organic base is used. More preferably ter. Amines such as trialkyl, triaryl, dialkylaryl or alkyl diaryl amines is used. Still more preferably trialkyl amine is used. Still more preferably diisopropyl ethyl amine is used.

Compound of formula XIV is contacted with a base in a suitable solvent to give a compound of formula I.

A solvent is selected from hydrocarbons, halogenated hydrocarbons, alcohols, ketones, esters, ethers, and the likes. Preferably solvent is selected from hydrocarbons. More preferably solvent is selected from aromatic hydrocarbons. Still more preferably the solvent is toluene.

Base is selected from alkali metal hydroxides, carbonates, bicarbonates, alkoxides, alkaline earth metal hydroxides and the like. Preferably the base is alkali metal hydroxides. More preferably base is potassium hydroxide.

The process is represented in scheme-6 herein below:

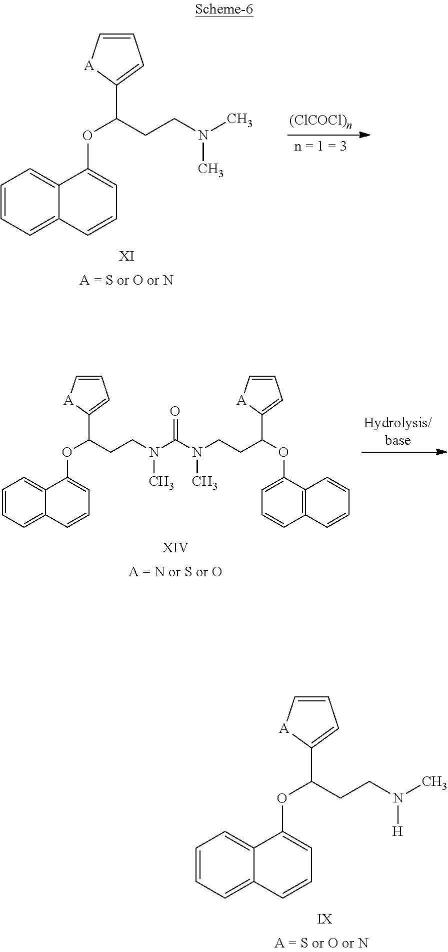

In yet another embodiment the method according to the present invention can be most advantageously applied for the preparation of compound (RS)N-methyl-3-(1-naphthyloxy)-3-(2-thienyl)-propylamine and its optical isomers preferably (+)-(S)—N-methyl-3-(1-naphthyloxy)-3-(2-thienyl)-propylamine known by the International Nonproprietary Name (INN) duloxetine of the formula II and IIA.

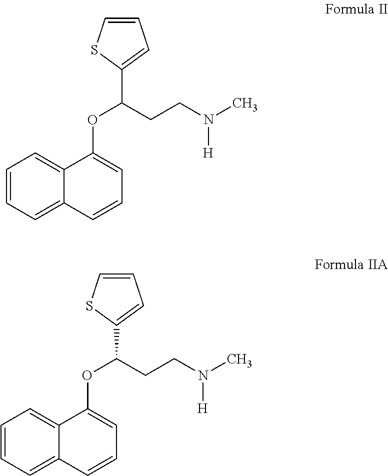 [SMILES: CNCCC(Oc1cccc2ccccc12)c1cccs1 ; CNCC[C@H](Oc1cccc2ccccc12)c1cccs1]

Therefore present invention includes a method for demethylating N,N-dimethylamino-3-(1-naphthanyloxy)-3-(2-thienyl)1-propanamine of formula II and its optically active isomer of formula IIA.

Process for making compounds of formulae II or HA comprises contacting a compound of formulae XV or XVA

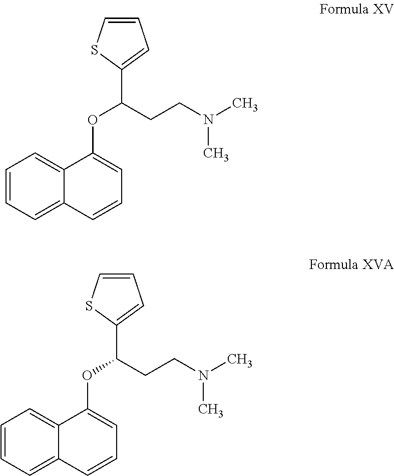 [SMILES: CN(C)CCC(Oc1cccc2ccccc12)c1cccs1 ; CN(C)CC[C@H](Oc1cccc2ccccc12)c1cccs1]

with a molecule of formula XIII Wherein n is 1 to 3

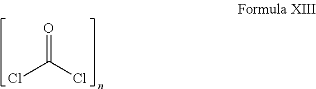

when n=1 the molecule of formula XIII is phosgene

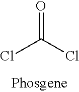

when n=2 the molecule of formula XIII is diphosgene

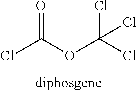

when n=3 the molecule of formula XIII is triphosgene

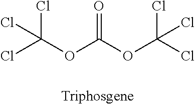 [SMILES: O=C(OC(Cl)(Cl)Cl)OC(Cl)(Cl)Cl]

in a suitable organic solvent in the presence of a base to give a novel compounds of formula XVI or XVIA

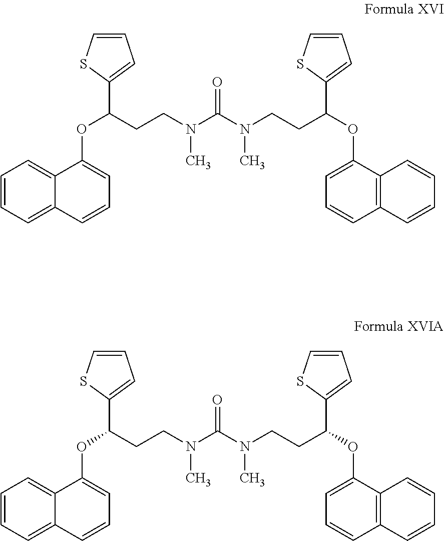 [SMILES: CN(CCC(Oc1cccc2ccccc12)c1cccs1)C(=O)N(C)CCC(Oc1cccc2ccccc12)c1cccs1 ; CN(CC[C@H](Oc1cccc2ccccc12)c1cccs1)C(=O)N(C)CC[C@@H](Oc1cccc2ccccc12)c1cccs1]

Compound of formula XIII represent phosgene, diphosgene and triphosgene. Preferably phosgene and more preferably triphosgene is used.

A solvent is selected from hydrocarbons, halogenated hydrocarbons, alcohols, ketones, esters, ethers, nitrogen atom based solvents such as triethylamine, pyridine and the likes. Preferably solvent is selected from hydrocarbons. More preferably solvent is selected from aromatic hydrocarbons. Still more preferably the solvent is toluene.

Inorganic or organic base can be used. Preferably organic base is used. More preferably ter. Amines such as trialkyl, triaryl, dialkylaryl or alkyl diaryl amines is used. Still more preferably trialkyl amine is used. Still more preferably diisoptropyl ethyl amine is used.

Compounds of formulae XVI or XVI A is contacted with a base in a suitable solvent to give a compd of formula II or II A.

A solvent is selected from hydrocarbons, halogenated hydrocarbons, alcohols, ketones, esters, ethers, and the likes. Preferably solvent is selected from hydrocarbons. More preferably solvent is selected from aromatic hydrocarbons. Still more preferably the solvent is toluene.

Base is selected from alkali metal hydroxides, carbonates, bicarbonates, alkoxides, alkaline earth metal hydroxides and the like. Preferably thbase is alkali metal hydroxides. More preferably base is potassium hydroxide.

The entire schematic representation for the preparation of duloxetine and its optically active isomers are shown in schemes 6 and 7 herein below.

Scheme II: Racemic duloxetine of formula II
Scheme III: Optically active duloxetine preferably (S) isomer of formula IIA

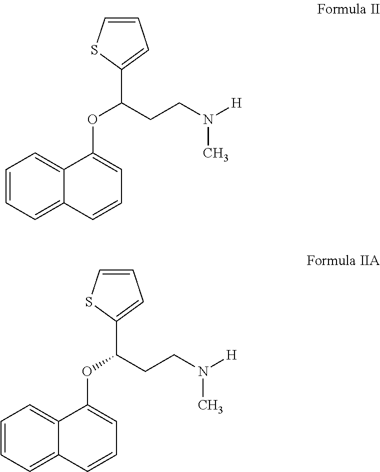 [SMILES: CNCCC(Oc1cccc2ccccc12)c1cccs1 ; CNCC[C@H](Oc1cccc2ccccc12)c1cccs1]

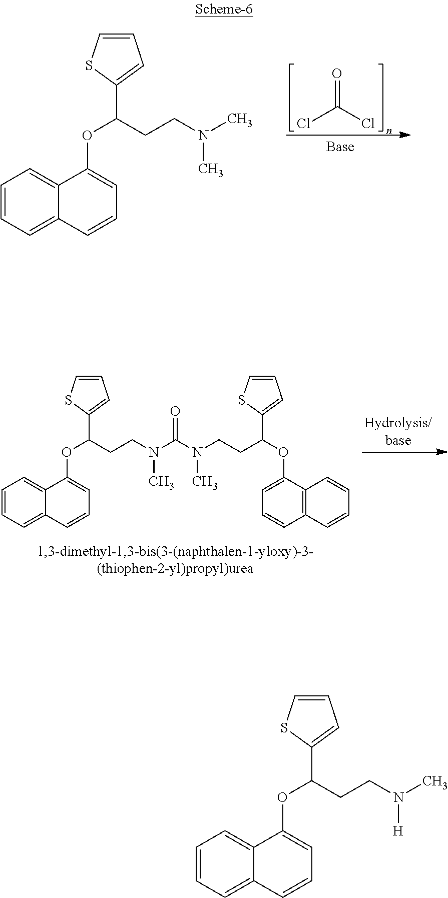

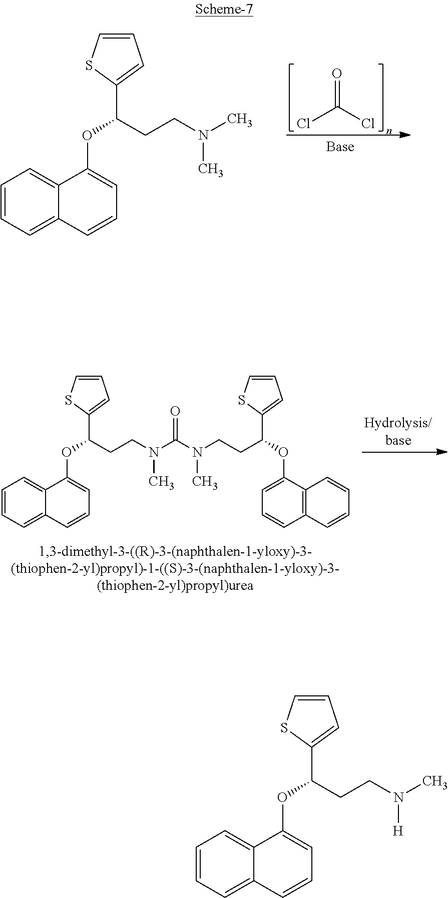

The base used for the hydrolysis of the compound XVI/XVIA is selected from the group containing inorganic bases selected from alkali metal hydroxide preferably potassium hydroxide.

It will be apparent to those skilled in the art that the various modifications and variations can be made in the present invention and specific examples provided herein without departing from the spirit or scope of the invention. Thus, it is intended that the present invention covers the modifications and variations of this invention that come within the scope of any claims and their equivalents.

The following examples are for illustrative purposes only and are not intended, or should they be interpreted to limit the scope of the invention.

### Example 1

Step A: Preparation of N,N-dimethylamino-3-(1-naphthanyloxy)-3-(2-thienyl)1-propanamine base: A clean and dry round bottom flask was charged with 70 grams of N,N-dimethylamino-3-(1-naphthanyloxy)-3-(2-thienyl)1-propanamine phosphate salt followed by the addition of 200 ml water. The pH of the above mixture was adjusted to 12 using 50% caustic lye at RT. N,N-dimethylamino-3-(1-naphthanyloxy)-3-(2-thienyl)1-propanamine so generated using 300 ml toluene in two times. The combined toluene layer was given a water wash and dried over sodium sulphate.

Step B: Preparation of N-methylamino-3-(1-naphthanyloxy)-3-(2-thienyl)1-propanamine base: The toluene layer obtained in step A was taken for cooling to reach at 0 to 5 degree Celsius to which 50 ml of diisopropylethylamine was charged followed by slow addition of triphosgene solution comprising 24 grams in 100 ml toluene keeping the temperature said above. The stirring was continued till the completion of the reaction and then temperature was brought up to 20 degree celsius. At the end of the reaction the mass was quenched using 10% caustic solution at 18-20 degree Celsius and stirring was continued for another 15 minutes. Toluene layer containing 1,3-dimethyl-1,3-bis(3-naphthalenyl-1-oxy)-3-(thiophenyl)-propyl urea so obtained was washed with water and then it was charged with 95 grams potassium hydroxide dissolved in 12.5 ml water. The contents were heated at 88-89 degree Celsius and continued the reaction completed. Reaction mass was cooled to 0 degree Celsius and pH was adjusted to 13 using 50% caustic lye. 180 ml fresh water was charged and mixture was stirred. Toluene layer so obtained was washed with water and dried. Toluene was removed under the reduced pressure to get the titled product.

Titled product is converted into its hydrochloride by reacting it with IPA/HCl as known in the practice. Duloxetine hydrochloride so formed is confirmed by NMR and IR.

NMR values for Duloxetine hydrochloride: 6 values are: 2.13 (m), 2.28 (m), 2.38 (s), 2.76 (m), 2.86 (m), 5.44 (t), 6.55 (m), 6.80-6.95 (m), 7.08 (t), 7.21 (t), 7.32 (d), 8.09 (d)

IR values: Values (cm⁻¹) 2800 (bs), 2370 (s), 1570(s), 1500 (m), 1398(m), 1240 (s)

### Example 2

Preparation of 1,3-dimethyl-3-((R)-3-(1-naphthalenyloxy)-3-(thiophen-2-yl)-propyl-((S)-3-(1-naphthalenyloxy)-3-(thiophen-2-yl)-propyl)urea: A clean and dry round bottom flask was charged with 70 grams of N,N-dimethylamino-3-(1-naphthanyloxy)-3-(2-thienyl)1-propanamine phosphate salt followed by the addition of 200 ml water. The pH of the above mixture was adjusted to 12 using 50% caustic lye at RT. N,N-dimethylamino-3-(1-naphthanyloxy)-3-(2-thienyl)1-propanamine so generated using 300 ml toluene in two times. The combined toluene layer was given a water wash and dried over sodium sulphate. The toluene layer so obtained was taken for cooling to reach at 0 to 5 degree Celsius to which 50 ml of diisopropylethylamine was charged followed by slow addition of triphosgene solution comprising 24 grams in 100 ml toluene keeping the temperature said above. The stirring was continued till the completion of the reaction and then temperature was brought up to 20 degree Celsius. At the end of the reaction the mass was quenched using 10% caustic solution at 18-20 degree Celsius and stirring was continued for another 15 minutes. Toluene layer containing 1,3-dimethyl-3-((R)-3-(1-naphthalenyloxy)-3-(thiophen-2-yl)-propyl-((S)-3-(1-naphthalenyloxy)-3-(thiophen-2-yl)-propyl)urea was washed with water to make free from alkali and dried over sodium sulphate. Toluene was removed under reduced pressure to get a syrupy liquid. The product so formed is confirmed by NMR.

NMR values for 1,3-dimethyl-3-((R)-3-(1-naphthalenyloxy)-3-(thiophen-2-yl)-propyl-((S)-3-(1-naphthalenyloxy)-3-(thiophen-2-yl)-propyl)urea: 6 values are: 2.37 (m), 2.6 (m), 3.1 (s), 3.7 (m), 6.1 (t), 7.02 (q), 7.13 (t), 7.31 (dd), 7.35 (t), 7.51 (d), 7.58 (d), 7.93 (d), 8.52 (d).

1. A process for preparation of compound of formula I 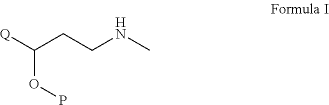
wherein Q and P independently represent substituted or unsubstituted aryl group selected from the group consisting of phenyl, naphthyl, pyridine, furanyl, pyranyl thienyl, further wherein the substituted aryl is substituted by a halogen, trifluoromethyl, a straight chain or a branched alkyl group containing 1 to 6 carbon atoms, —O-alkyl group containing straight chain or branched C₁-C₆ alkyl group, an alkoxy group comprises a straight chain or branched alkyl group having 1 to 6 carbon atoms or combinations thereof;
comprising the steps of:
a) contacting a compound of formula IA 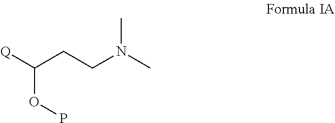
wherein substituents Q and P are same as in compound of formula I
with a compound of formula XIII 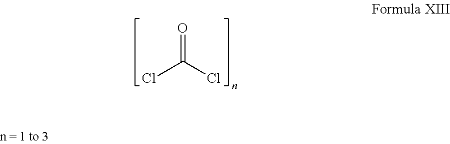
in an organic solvent in the presence of a base to give a compound of formula XII 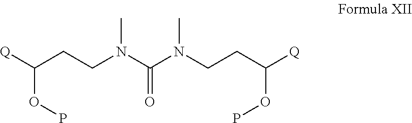
b) contacting compound of formula XII obtained in step 1a with a base in an organic solvent to give a compound of formula I.

2. The process of claim 1, wherein the organic solvent is selected from the group consisting of hydrocarbons, halogenated hydrocarbons, alcohols, ketones, esters, ethers, nitrogen atom based solvents comprising triethylamine, pyridine, or mixtures thereof.

3. The process of claim 1, wherein the solvent is selected from aromatic hydrocarbons.

4. The process of claim 1, wherein in step 1a the base is selected from the group consisting of trialkyl, triaryl, dialkylaryl or alkyl diaryl amines or mixtures thereof.

5. The process of claim 1 wherein in step 1b the base is selected from the group consisting of alkali metal hydroxides, carbonates, bicarbonates, alkoxides, and alkaline earth metal hydroxides.

6. The process of claim 5, wherein the base is alkali metal hydroxide.

7. The process of claim 1, wherein “P” is naphthyl and “Q” is thienyl.

8-12. (canceled)

13. The process according to claim 1, wherein said compound of formula I is selected from the group consisting of duloxetine, (S) duloxetine, fluoxetine, tomoxetine, atomoxetine and nisoxetine.

14. The process of claim 1 for the preparation of compound of formula IIA 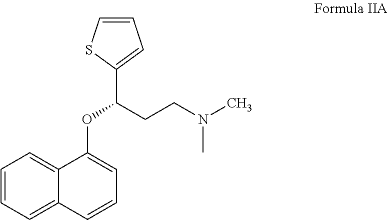 [SMILES: CN(C)CC[C@H](Oc1cccc2ccccc12)c1cccs1]
comprising the steps of:
a) contacting a compound of formula XV A 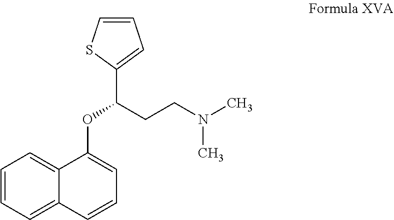 [SMILES: CN(C)CC[C@H](Oc1cccc2ccccc12)c1cccs1]
with a compound of formula XIII
in the organic solvent in the presence of the base to give a compound of formula XVI A 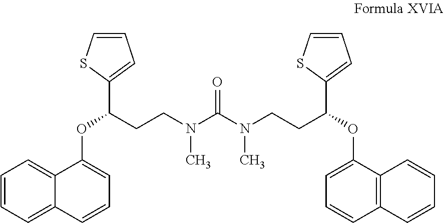 [SMILES: CN(CC[C@H](Oc1cccc2ccccc12)c1cccs1)C(=O)N(C)CC[C@@H](Oc1cccc2ccccc12)c1cccs1]
b) contacting the compound of formula XVI A obtained in step a with the base in the organic solvent to give the compound of formula IIA.

15. The process of claim 14, wherein the organic solvent is selected from the group consisting of hydrocarbons, halogenated hydrocarbons, alcohols, ketones, esters, ethers, amines or mixture thereof.

16. The process of claim 15, wherein the solvent is selected from aromatic hydrocarbons.

17. The process of claim 14, wherein in step 14a, the base is selected from the group consisting of trialkyl, triaryl, dialkylaryl or alkyl diaryl amines or mixtures thereof.

18. The process of claim 14, wherein in step 14b, the base is selected from the group consisting of alkali metal hydroxides, carbonates, bicarbonates, alkoxides, and alkaline earth metal hydroxides.

19. The process of claim 18, wherein the base is alkali metal hydroxide.

20. A compound of formula XII, 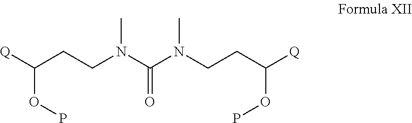
wherein, wherein Q and P independently represent substituted or unsubstituted aryl group selected from the group consisting of phenyl, naphthyl, pyridine, furanyl, pyranyl thienyl, further wherein the substituted aryl is substituted by a halogen, trifluoromethyl, a straight chain or a branched alkyl group containing 1 to 6 carbon atoms, —O-alkyl group containing straight chain or branched C₁-C₆ alkyl group, an alkoxy group comprises a straight chain or branched alkyl group having 1 to 6 carbon atoms or combinations thereof.

21. A compound which is selected from the group consisting of

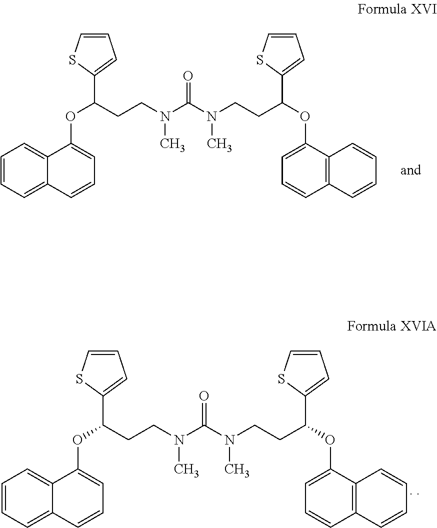 [SMILES: CN(CCC(Oc1cccc2ccccc12)c1cccs1)C(=O)N(C)CCC(Oc1cccc2ccccc12)c1cccs1 ; CN(CC[C@H](Oc1cccc2ccccc12)c1cccs1)C(=O)N(C)CC[C@@H](Oc1cccc2ccccc12)c1cccs1]

22. The Compound of claim 20 wherein, said compound is used as an intermediate for preparation of a compound of Formula I,

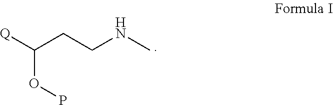

23. The compound of claim 21 wherein, said compound is used as an intermediate for preparation of duloxetine and (S) duloxetine.
